(19) United States
(12) Patent Application Publication
(10) Pub. No.: US 2008/0114335 A1
(43) Pub. Date: May 15, 2008

(54) Medical Device Guide

(76) Inventors: William Flickinger, Lino Lakes, MN (US); Amy Raatikka, Plymouth, MN (US); Scott Klimek, Maple Grove, MN (US); Nathan Kastelein, St. Louis, MO (US); Gareth T. Munger, St. Louis, MO (US); Carlo Pappone, Milan (IT); Gabrielle Vicedomini, Milan (IT)

Correspondence Address: HARNESS, DICKEY, & PIERCE, P.L.C, 7700 BONHOMME, STE 400, ST. LOUIS, MO, 63105, US

(21) Appl. No.: 11/844,105

(22) Filed: Aug. 23, 2007

Related U.S. Application Data
(60) Provisional application No. 60/839,620, filed on Aug. 23, 2006.

(51) Int. Cl. A61M 25/00

(52) U.S. Cl. 604/523

(57) ABSTRACT

A medical device guide having an inner member having a proximal end, a distal end, and at least one lumen therein. An outer member surrounds the proximal portion of the inner member such that the distal end of the inner member projects beyond the distal end of the outer member, the portion of the inner member being sufficient flexible to flex with a guide wire extending through the lumen. The outer member is sufficiently stiff that the proximal end of the medical device guide can be engaged by a driven by a medical device advancer.

### CROSS-REFERENCE TO RELATED APPLICATION

This application claims priority to prior U.S. patent application Ser. No. 60/839,620, filed Aug. 23, 2006, the entire disclosure of which is incorporated herein by reference.

### FIELD OF THE INVENTION

The present disclosure relates generally to guides for facilitating the navigation of medical devices such as guide wires and catheters through the body.

### BACKGROUND

The statements in this section merely provide background information related to the present disclosure that is not necessarily prior art.

Interventional medical procedures are medical procedures that employ medical devices that are navigated through body lumens and cavities such as the subjects vasculature. Because of improvements in these types of devices, and improvements in the ability to navigate these devices, interventional medical procedures continue to increase in popularity, placing increasing demands on faster and safer navigation techniques. Initially medical devices were manipulated by hand through the vasculature. Various mechanical methods of steering such devices have been developed to improve navigation. Most recently remote navigation techniques, such as remote magnetic navigation, have bee developed.

Despite these advances, there is still room for improvement in the automated navigation of medical devices, particularly in the automated navigation through the cardiac vasculature and the coronary sinus in particular. SUMMARY

The present invention relates to a guide for facilitating the navigation of medical devices, and particularly guide wires in the body.

A preferred embodiment of medical device guide in accordance with the principles of this invention comprises an inner member, having a proximal end and a distal end, and a lumen therethrough for receiving a medical device such as guide wire. An outer member is disposed over the proximal end portion of the inner member. The portion of the inner member that extends beyond the distal end is highly flexible and accommodative of the medical device extending through the lumen. The outer member can serve to stiffen the proximal end portion of the device and/or increase its outer diameter to facilitate advancing the wire guide, particularly with automated advancers.

In addition to the lumen for the medical device, in the preferred embodiment, there is an additional lumen for electrical leads to electrodes on the distal end of the inner member, and at least one, and preferably at least two additional lumens for imaging contrast media.

The medical device guide of the preferred embodiment can be introduced over a guide wire or other medical device and advanced through the vasculature. The outer member can provide sufficient stiffness and size to allow the wire guide to be advanced with an automated advancer, while the flexible distal portion of the inner member flexes with the medical device, and provides a smooth transition relative to the stiffer proximal end of the guide. In addition the distal end of the inner member can carry electrodes or magnetically responsive elements for enabling magnetic navigation.

Embodiments of the guide can be configured for rapid exchange, with a radial slit extending along the length of the guide to allow the guide to be pulled off of a medical device disposed in the lumen of the inner member. Alternatively the radial slit can be provided only in the inner member, and outer member can be removable.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a side elevation view of one embodiment of a wire guide of the present application;

FIG. 2 is a perspective view of a portion of the wire guide shown in FIG. 1;

FIG. 3 is a cross-sectional view of the first embodiment of a wire guide;

FIG. 4 is a cross-sectional view of a second embodiment of a wire guide;

FIG. 5 is a cross-sectional view of a third embodiment of a wire guide;

FIG. 6 is a cross-sectional view of a fourth embodiment of a wire guide;

FIG. 7 is a cross-sectional view of a fifth embodiment of a wire guide; and

FIG. 8 is a cross-sectional view of a sixth embodiment of a wire guide

Corresponding reference numerals indicate corresponding parts throughout the several views of the drawings.

### DETAILED DESCRIPTION

The following description is merely exemplary in nature and is not intended to limit the present disclosure, application, or uses. It should be understood that throughout the drawings, corresponding reference numerals indicate like or corresponding parts and features.

The present disclosure describes various embodiments of a wire guide that is adapted to be received over a conventionally or magnetically-guided guide wire, for use in interventional medical procedures. One or more embodiments of a guide wire generally comprise an outer member and an inner member disposed within the outer member. The inner member extends a distance beyond the distal end portion of the outer member. The inner member further includes at least one lumen extending therethrough, where the inner member is capable of receiving a guide wire within the at least one lumen. The wire guide comprising the outer member and inner member is capable of being advanced over a guide wire received within a lumen of the inner member of the guide wire. One or more lumens could be used for the delivery of contrast materials, pharmaceuticals, gas, saline or biological agents, such as stem cells or other tissues. At least one lumen may exit the device along the long axis or it may exit the device substantially off-axis.

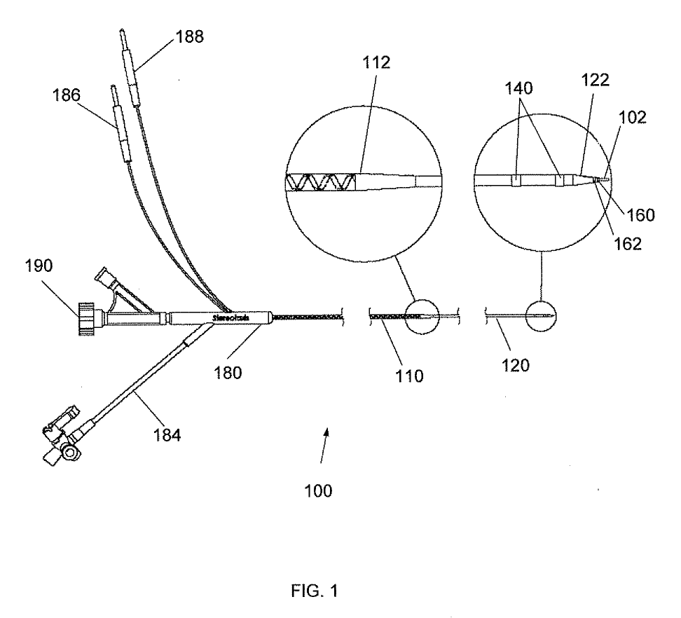

One first embodiment of a wire guide is generally shown in FIG. 1 at 100. As shown and described in this preferred embodiment, the medical device guide is particular adapted for use with a medical guide wire, but this invention is not so limited and can be uses with other medical devices including for example, catheters, endoscopes, and electrical leads. The wire guide 100 comprises an outer tubular member 110 having a lumen in which an inner tubular member 120 is disposed. The inner member 120 has a proximal end and a distal end 122, and at least one lumen (not shown in FIG. 1) therein capable of receiving a medical device such as a guide wire 102.

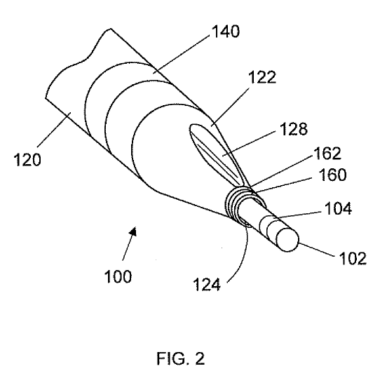

The inner tubular member 120 extends beyond the distal end portion 112 of the outer tubular member 110. The inner tubular member 120 has a flexible distal end portion 122, and may include at least one magnetically responsive element 140 disposed around or embedded within the tubular member 120 near the distal end portion 122. The inner member 120 is preferably made of a material that is sufficiently flexible to pass easily over the guide wire 102 without disrupting the guide wire, yet is sufficiently stiff to be advanced through the vasculature. The distal end 122 of the inner member 120 preferably has a smooth, rounded, tapered configuration to facilitate advancement through the vasculature. One or more of the lumens 128 may open through the tapering distal end 122, as shown in FIG. 2.

The proximal end of the outer member 110 is preferably connected to or received within a manifold 180. The manifold 180 includes a rotating hemostasis valve 190, and may optionally include a second valve connection 192. The wire guide 100 has a working length of about 120 centimeters (47.25 inches) from the distal end 122 of the inner tubular member 120 to the distal end of the manifold 180. The manifold 180 may further include a tap 184 to provide for introduction of imaging or contrast agents, or other therapeutic agents, into a lumen of the inner member 120. One or more conductive wires 186 and 188 are provided that extend through at least one lumen in the inner member 120 to an electrode 160 near the distal end. The conductive wires 186 and 188 project or extend from the manifold 180, to enable sensing of electrical activity of tissues that the electrode 160 may contact. The inner member 120 further includes at least one lumen in which a mechanically or magnetically navigable guide wire 102 may be received. The wire guide 100 may accordingly be placed onto and advanced over a guide wire 102 into a subject's body.

It should be noted that the wire guide 100 may be placed over the proximal end of a guide wire 102 (the distal end of which has been navigated to a target area within a subject's body) and advanced over the wire guide 102 into a subject's body towards the target area. The wire guide 100 may also be inserted and advanced over the proximal end of a guide wire 102, such that the distal ends of the wire guide 100 and guide wire 102 may be navigated and advanced together through a subject's body. In either situation, the wire guide 100 is capable of providing support for the guide wire 102, which may be preferably be extended beyond the distal end of the inner member 120 up to 5 centimeters. The wire guide 100 is also capable of providing navigational assistance to the guide wire, by guiding the extended end of the guide wire 102 towards a target area within a subject's body. The wire guide 100 may also be advanced over a guide wire that has been previously navigated to a target area within a subject's body, to bring an electrode 160 of the wire guide 100 to the target area for sensing electrophysiological activity at various tissue locations, or to perform ablation at various points. The wire guide 102 may be, for example, a magnetically navigable guide wire having one or more magnetically responsive elements disposed on the distal end portion. Such a wire guide 102 may be guided through a subject's body by applying a magnetic field to cause the magnetically responsive elements 104 to substantially align with the direction of the magnetic field to orient the distal end portion of the guide wire in a desired direction.

Referring to FIG. 2, the first embodiment includes an inner tubular member 120 having a tapered distal end portion 122. The inner tubular member 120 has at least one lumen 124 capable of receiving a guide wire 102 therein. The inner tubular member 120 further includes at least one lumen 128 therein for delivery of fluids therethrough. The inner tubular member 120 is preferably made of a material having an effective amount of stiffness for enabling the inner tubular member to be advanced over a guide wire 102 received within the lumen 124 in the inner tubular member 120. The inner tubular member 120 may further comprise at least one magnetically responsive member 140 near the distal end portion 122. The at least one magnetically responsive member 140 disposed on the distal end portion 122 of the inner member 120 is capable of being substantially aligned with the direction of an applied magnetic field, to cause the distal end portion 122 of the inner member 120 to be oriented in a desired direction.

The inner member 120 may further include one or more electrodes 160 on the distal end portion 122 of the inner tubular member 120, which may be connected to one or more conductive wires extending through at least one lumen 126 in the inner member 120. The electrode 160 is preferably made of a conductive metallic material for enabling sensing of electrical activity and the pacing of nerve or muscle tissue. The electrode 160 may also be made of material suitable for improving ultrasound visualization, or made of a radiopaque material for improving visualization of the electrode under fluoroscopy imaging. By retracting the guide wire 102 within the lumen 124 of the inner member 120, the wire guide 100 may then be navigated to accurately position the one or more electrodes 160 on the distal tip to permit sensing electrical activity of the tissue at various locations. At least one electrode may further be used as a localization sensor for non-fluoroscopy localization, such as for example with an electrically-based localization system.

In this preferred embodiment, the inner member 120 has two electrodes adjacent to or near the distal end 122 as shown in FIG. 2, so that the wire guide can be used to measure local electrical signals and/or apply pacing signals and/or to apply ablative energy to adjacent tissue. The two electrodes 160 and 162 are preferably bands extending around the circumference of the distal end of the inner member 120, spaced from each other. While two electrodes are shown and described, the device need not have any electrodes, Furthermore, the device could have a single electrode and operate in a monopole configuration, or could have multiple electrodes and operate in multi pairs or in mulitpole modes. Lead wires 186 and 188 extend from electrodes 160 and 162, respectively, through a lumen, and exit the lumen near the proximal end of the outer member 120. The lead wires 186 and 188 terminate in connectors adjacent the proximal end of the wire guide 100 for connecting the electrodes 160 and 162 to signal measuring, pacing signal generating, and/or RF generating equipment.

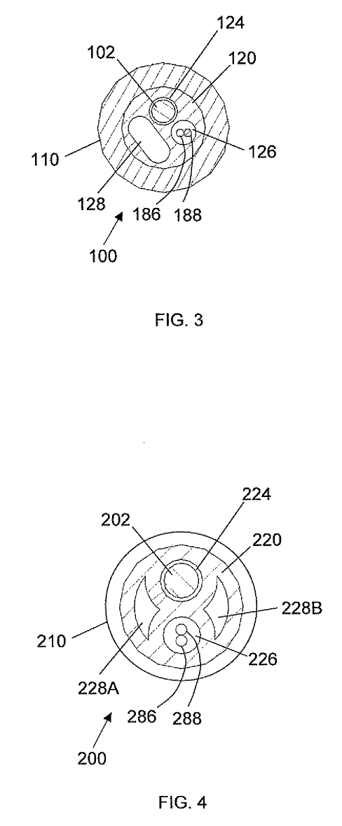

Referring to FIG. 3, a cross-section of the wire guide 100 of the first embodiment is shown. The wire guide 100 is preferably a 4-French diameter size, or 1.333 millimeters (0.050 inches ) in diameter. The outer tubular member 110 is preferably thermally bonded or over-molded onto the inner tubular member 120, but may alternatively be bonded by adhesive, fasteners, or other means suitable for securing the inner and outer members. The outer member 110 is made of a polymer or rubber material having a durometer in the range of 35 to 63 Shore-D hardness, sufficient to resist crushing of the outer tubular member 110 by an advancing device that frictionally engages the outer surface of the outer tubular member 110. The outer tubular member 110 may also include a reinforcing fiber dispersed within the material, or a coiled wire or braid embedded within the walls of the outer tubular member 110, or a combination thereof. The reinforcing structure also enhances frictional engagement with the wire guide 100. This allows in the wire guide 100 to be driven by an automated advancer mechanism engaging the proximal end portion of the wire guide. An example of such an automated advancer mechanism is the CAS system available from Stereotaxis, Inc., St. Louis, Mo., and disclosed in U.S. patent application Ser. No. 10/138,710, filed May 3, 2002, for System and Methods for Advancing a Catheter, or U.S. patent application Ser. No. 10/858,485, filed Jun. 1, 2004, for System and Methods for Medical Device Advancement. The reinforcement of the outer member 110 helps prevent the lumens in the inner member 120 from being crushed, as well as medical devices in those lumens from being damaged. The reinforcement of the outer member 110 further helps the outer member 110 to maintain structural integrity when interfaced with a mechanical advancer unit that might apply enough force to crush the outer member if it was otherwise not reinforced. The outer member 110 also increases the outer diameter of the proximal end of the wire guide, which can also facilitate advancement of the wire guide using an automated advancer. In the case of use with a magnetic navigation system, the reinforcing material may be made of non-ferrous materials to eliminate its interaction with strong magnetic fields. In other embodiments, the outer member acts as a rack and pinion with the drive wheels of the mechanical advancer.

In the first embodiment, the inner member 120 comprises one or more lumens extending through at least a portion of the inner member 120. The inner member 120 preferably includes at least three lumens 124, 126, and 128. Lumen 124 extends through the distal end 122 of the inner member 120, and is capable of receiving therein a mechanically or magnetically navigable guide wire with a nominal diameter of about 1 millimeter (0.014 inches). Lumen 126 extends through at least a portion of the inner member 120, and is capable of receiving therein one or more conductive wires 186 and 188, which extend from the proximal end of the wire guide 100 to the electrode 160. At least one lumen 128 extends through the distal end 122 of the inner member 120, and is capable of delivering imaging or contrast agents, saline, gas, biologically active agents, or pharmaceutical or therapeutic agents there through. While the at least one lumen 128 is shown with an oval or oblong cross-section, the at least one lumen 128 may alternatively comprise two separate lumens for enabling separate delivery of different agents through each lumen. In the first embodiment, the at least one lumen 128 comprises one lumen having a cross-sectional area of at least 1.94 millimeters² (0.003 inches²), where the wire guide 100 has an outer diameter of about 1.27 millimeters (0.050 inches). The proximal end of the wire guide 100 is designed to enable access to each lumen without impacting the other lumens, particularly the guide wire lumen and the contrast injection lumen, where one would not wish to mix the contents of the two lumens.

In a second embodiment of a wire guide 200 as shown in FIG. 4, the wire guide 200 comprises an outer member 210 having an inner member 220 that extends beyond the distal end of the outer member 210 similar to that of the first embodiment. The inner member 220 preferably includes at least three lumens 224, 226, and 228. Lumen 224 extends through the distal end 222 of the inner member 220, and is capable of receiving therein a mechanically or magnetically navigable guide wire with a nominal diameter of about 1 millimeter (0.014 inches). Lumen 226 extends through at least a portion of the inner member 220, and is capable of receiving therein one or more conductive wires 286 and 288, which extend from the proximal end of the wire guide 200 to an electrode (not shown). At least one lumen 228 extends through the distal end 222 of the inner member 220, and is capable of delivering fluids therethrough. At the proximal end of the wire guide 200, a fluid tight connection can be made to introduce a fluid, such as a liquid imaging contrast agent, into the lumen 228 of the wire guide. The contrast agent can make the wire guide easier 200 to see in images, and/or inject the contrast media into the body lumen in which the wire guide is disposed to make the lumen easier to see in images. As shown in FIG. 4 there are preferably two lumens 228A and 228B, to increase the volume of contrast agent delivered via the wire guide. It should be noted that the lumens may be used to deliver imaging or contrast agents, saline, gas, biologically active agents, or pharmaceutical or therapeutic agents through the distal end of the guide to a target area within a subject's body. The two areas of the at least one lumen 228 are in communication with each other, such that the areas function as one lumen. The at least one lumen 228 may alternatively comprise two separate lumens for enabling separate delivery of different agents through each lumen. In the second embodiment, the cross-sectional area of the lumen 228 is at least 1.94 millimeters² (0.003 inches²), where the wire guide 200 has an outer diameter of about 1.27 millimeters (0.050 inches).

In a third embodiment of a wire guide 300 as shown in FIG. 4, the wire guide 300 comprises an outer member 310 having an inner member 320 that extends beyond the distal end of the outer member 310 similar to that of the first embodiment. The inner member 320 preferably includes at least four lumens 324, 326, 327 and 328. Lumen 324 extends through the distal end 322 of the inner member 320, and is capable of receiving therein a mechanically or magnetically navigable guide wire with a nominal diameter of about 1 millimeter (0.014 inches). Lumen 326 extends through at least a portion of the inner member 320, and is capable of receiving therein one or more conductive wires 386 and 388, which extend from the proximal end of the wire guide 300 to an electrode (not shown). A lumen 327 extends through at least a portion of the inner member 320, and is capable of receiving therein a pull wire 398 that may be terminated in a handle or other interface to mechanical actuators for enabling the steering of the wire guide 300. It should be noted that the wire guide of this embodiment may be navigated through both magnetic navigational control and mechanical navigational control means. The wire guide 300 includes at least one lumen 328 that extends through the distal end 322 of the inner member 320, which is capable of delivering imaging or contrast agents, saline, gas, biologically active agents, or pharmaceutical or therapeutic agents there through.

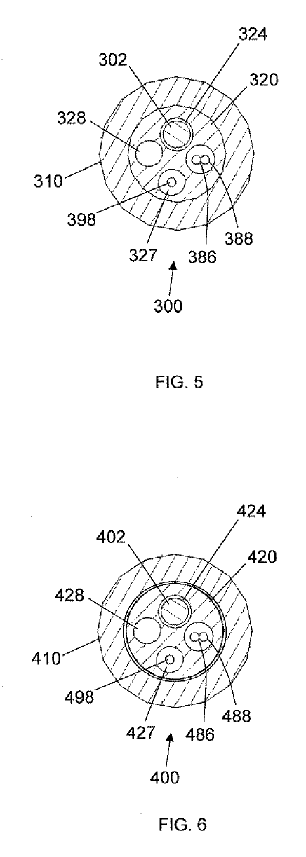

In a fourth embodiment of a wire guide 400 as shown in FIG. 5, the wire guide 400 comprises an outer member 410 having a lumen therein in which an inner member 420 is slidably disposed. The inner member 420 is accordingly capable of being extended beyond the distal end of the outer member 310 to any desired set length. The inner member 420 preferably includes at least four lumens 424, 426, 427 and 428. Lumen 424 extends through the distal end 422 of the inner member 420, and is capable of receiving therein a mechanically or magnetically navigable guide wire with a nominal diameter of about 1 millimeter (0.014 inches). Lumen 426 extends through at least a portion of the inner member 420, and is capable of receiving therein one or more conductive wires 486 and 488, which extend from the proximal end of the wire guide 400 to an electrode (not shown). A lumen 427 extends through at least a portion of the inner member 420, and is capable of receiving therein a pull wire 498 that may be terminated in a handle or other interface to mechanical actuators for enabling the steering of the wire guide 400. It should be noted that the wire guide of this embodiment may be navigated through both magnetic navigational control and mechanical navigational control means. The wire guide 400 includes at least one lumen 428 that extends through the distal end 422 of the inner member 420, which is capable of delivering imaging or contrast agents, saline, gas, biologically active agents, or pharmaceutical or therapeutic agents there through.

It should be noted that in the construction of a wire guide, such as the first embodiment of wire guide for example, the inner member 120 and the outer member 110 can be separate pieces, or the inner member 120 and the outer member 110 can be a single piece, either formed monolithically, such as by molding, or formed in multiple pieces and secured together. The outer member may be of a diameter size that enables it to be used with standard sheaths available on the market. In a preferred embodiment, the inner member can have an outer diameter of between 2 F and 10 F in standard French size units (between 0.66 mm and 3.33 mm), while the outer member can have an inner diameter between 3 F and 11 F (1 mm and 3.66 mm).

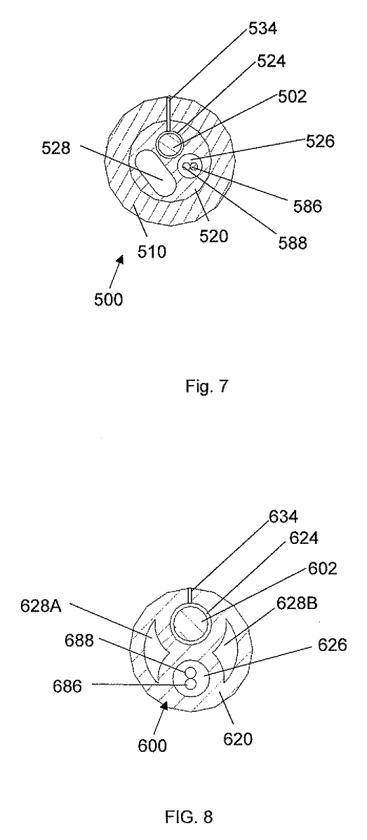

A fifth embodiment of a wire guide is indicated generally as 500 in FIG. 7. As shown in FIG. 7, the wire guide comprises an outer member 510 and an inner member 520, with one or more lumens extending through at least a portion of the inner member 520. The inner member 520 preferably includes at least three lumens 524, 526, and 528. Lumen 524 extends through the distal end of the inner member 520, and is capable of receiving therein a mechanically or magnetically navigable guide wire with a nominal diameter of about 1 millimeter (0.014 inches). Lumen 526 extends through at least a portion of the inner member 520, and is capable of receiving therein one or more conductive wires 586 and 588, which extend from the proximal end of the wire guide 500 to the electrode. At least one lumen 528 extends through the distal end of the inner member 520, and is capable of delivering imaging or contrast agents, saline, gas, biologically active agents, or pharmaceutical or therapeutic agents there through. While the at least one lumen 528 is shown with an oval or oblong cross-section, the at least one lumen 528 may alternatively comprise two separate lumens for enabling separate delivery of different agents through each lumen. In the fifth embodiment, the at least one lumen 528 comprises one lumen having a cross-sectional area of at least 1.94 millimeters² (0.003 inches²), where the wire guide 500 has an outer diameter of about 1.27 millimeters (0.050 inches). The proximal end of the wire guide 500 is designed to enable access to each lumen without impacting the other lumens, particularly the guide wire lumen and the contrast injection lumen, where one would not wish to mix the contents of the two lumens.

In a sixth embodiment of a wire guide 600 as shown in FIG. 8, the wire guide 600 comprises an inner member 620 that preferably includes at least three lumens 624, 626, and 628. Lumen 624 extends through the distal end of the inner member 620, and is capable of receiving therein a mechanically or magnetically navigable guide wire with a nominal diameter of about 1 millimeter (0.014 inches). Lumen 626 extends through at least a portion of the inner member 620, and is capable of receiving therein one or more conductive wires 686 and 688, which extend from the proximal end of the wire guide 600 to an electrode (not shown). At least one lumen 628 extends through the distal end of the inner member 620, and is capable of delivering fluids therethrough. At the proximal end of the wire guide 600, a fluid tight connection can be made to introduce a fluid, such as a liquid imaging contrast agent, into the lumen 228 of the wire guide. The contrast agent can make the wire guide easier 600 to see in images, and/or inject the contrast media into the body lumen in which the wire guide is disposed to make the lumen easier to see in images. As shown in FIG. 4 there are preferably two lumens 628A and 628B, to increase the volume of contrast agent delivered via the wire guide. It should be noted that the lumens may be used to deliver imaging or contrast agents, saline, gas, biologically active agents, or pharmaceutical or therapeutic agents through the distal end of the guide to a target area within a subject's body. The two areas of the at least one lumen 628 are in communication with each other, such that the areas function as one lumen. The at least one lumen 628 may alternatively comprise two separate lumens for enabling separate delivery of different agents through each lumen. In the second embodiment, the cross-sectional area of the lumen 628 is at least 1.94 millimeters² (0.003 inches²), where the wire guide 600 has an outer diameter of about 1.27 millimeters (0.050 inches).

Embodiments of the wire guide can also be adapted for use in a rapid exchange mode. As shown in the embodiment in FIG. 7, a radial slit 534 can extend through the inner and outer members 520 and 510, to the lumen 524, so that wire guide 500 can be removed from a medical device such as guide wire 502. As shown in FIG. 8. in some constructions of a wire guide embodiment, the slit 634 extends only through the inner member 620. In these embodiments, the outer member 610 is first removed form the inner member 620, and the inner member 620 is pulled from around the guide wire 602.

Further, in one preferred embodiment the wire guide may have magnets in the tip to enable the wire guide to be steered with a magnetic navigation system. In an alternate preferred embodiment, the wire guide may have at least one pull wire running the majority of its length and terminating in a handle or other interface to enable the wire guide to be steered manually or using mechanical actuators. In still another preferred embodiment, the wire guide may incorporate a balloon on the distal end. In one embodiment, the balloon could be used for the obstruction of blood flow to enhance the contrast during angiography or to deliver a stent. In another preferred embodiment, the wire lumen is of sufficient diameter to deliver a pacing or other electrical lead.

### Operation

In operation, a guide wire 100 is introduced into the lumen 124 of the inner member 120 and the combined wire guide 100 and guide wire 102 are introduced in the subject's vasculature, with the wire guide protecting the guide wire. The distal end of the guide wire 102 is advanced about 4 or 5 cm from the distal end of the wire guide 100, and the guide wire and wire guide are secured at the proximal end of the wire guide. The guide wire 102 can be a conventional guide wire that is navigated manually, but is preferably a magnetically navigable guide wire that can be oriented in a desired direction with the application of magnetic field from an external source magnet. Such magnetically navigable guide wires are available from Stereotaxis, Inc., St. Louis, Mo. In some embodiments, magnetically responsive elements 140 can be incorporated near the distal end 122 of the inner member 120, instead of or in addition to magnetically responsive elements provided on the guide wire 102, so that the distal end of the inner member 120 is magnetically orientable. The larger and stiffer outer member 110 is adapted to be engaged by, an driven by, an automated advancer unit, so that the advance of the wire guide 100 and guide wire 102 therein can be remotely controlled.

The wire guide 100 and guide wire combination are advanced by orienting the distal end of the guide wire and advancing the devices using the automated advancer. This arrangement allows the physician to be positioned away form the operating region, reducing exposure to imaging x-rays. The tip of the wire is navigated past the site of interest until the distal end of the wire guide 100 is at the site of interest.

When in the desired location at the site of interest, the electrodes 160 and 162 can be used to measure local electrical activity. Alternatively or additionally, the electrodes 160 and 162 can be used to apply pacing signals or ablative energy to selectively ablate tissue adjacent the electrodes. Typically, after preliminary measurements are taken, the wire guide 102 is removed from the guide wire 110. The wire guide can be withdrawn over the proximal end of the wire, or in the case of the embodiments shown in FIGS. 7 or 8, the wire guide can be stripped from the guide wire through the slit allowing the guide wire to exit the lumen of the inner member. In some embodiments such as in FIG. 7, the slit 534 extends through both the inner member 520 and the outer member 510 of the wire guide. In other embodiments, such as in FIG. 8, the slit 534 extends just through the inner member 620, and the outer member 610 must first be removed from the inner member. Once it is confirmed that the wire guide 100 has delivered the guide wire 102 to an appropriate location, a pacing lead or other device can then be advanced over the guide wire 102 into proper position.

1. A medical device having an outer member and an inner member disposed within the outer member, and having at least one lumen therein, the inner member extending beyond the end of the outer member, and being capable of receiving a guide wire within the at least one lumen; wherein the inner member is capable of being advanced over a guide wire that is received within the at least one lumen of the inner member.

2. The medical device of claim 1, wherein the inner member is made of a polymer-based material having a durometer of sufficient hardness to enable the inner member to be extended over and follow a guide wire received within the lumen of the inner member.

3. (canceled)

4. The medical device of claim 1, wherein the outer member is of sufficient strength to withstand forces applied by an advancing apparatus.

5. The medical device of claim 1, wherein the outer member is made of a polymer that is reinforced with one of a fiber, braid, or webbing.

6. (canceled)

7. (canceled)

8. The medical device of claim 7 further comprising a conductor connected to the electrode, which conductor extends through the inner member to the proximal end portion, for enabling sensing of electrical activity of tissue in contact with the electrode.

9-13. (canceled)

14. A wire guide for guiding a guide wire, comprising:
an inner tubular member having at least one lumen capable of receiving a guide wire therein and at least one lumen for delivery of fluids therethrough, the inner tubular member being made of a material having an effective amount of stiffness for enabling the inner tubular member to be advanced over a guide wire received within the inner tubular member;
an outer tubular member capable of receiving an inner tubular member therein, wherein the inner tubular member is capable of extending beyond the end of the outer tubular member;
at least one magnetically responsive member disposed on the distal end portion of the inner member, which magnetically responsive member is capable of being substantially aligned with the direction of an applied magnetic field to cause the distal end portion of the inner member to be oriented in a desired direction; and
an electrode on the distal end portion of the inner tubular member.

15. The medical device of claim 14, wherein the outer member is of sufficient strength to resist crushing by an advancing apparatus.

16. The medical device of claim 14, wherein the outer member is made of a polymer that is reinforced with one of a fiber, braid, or webbing, for improving resistance to crushing of the outer member .

17. (canceled)

18. (canceled)

19. (canceled)

20. (canceled)

21. (canceled)

22. (canceled)

23. A medical device guide having an inner member having a proximal end, a distal end, and at least one lumen therein, and an outer member surrounding the proximal portion of the inner member such that the distal end of the inner member projects beyond the distal end of the outer member, the portion of the inner member being sufficient flexible to flex with a guide wire extending through the lumen, the outer member being sufficiently stiff that the proximal end of the medical device guide can be engaged to be driven by a medical device advancer.

24. The medical device guide according to claim 23 further comprising an electric lead lumen extending at least partially through the inner member, and wherein there is at least one electrode adjacent the distal end of the inner member, the at least one electrode being connected to a lead wire that extends through electric lead lumen.

25. The medical device guide according to claim 23 further comprising at least one fluid lumen extending substantially through the inner member.

26. (canceled)

27. (canceled)

28. The medical device of claim 23, wherein the inner member is made of a polymer-based material having a durometer of sufficient hardness to enable the inner member to be extended over and follow a guide wire received within the lumen of the inner member.

29. (canceled)

30. The medical device of claim 23, wherein the outer member is of sufficient strength to withstand forces applied by an advancing apparatus.

31. The medical device of claim 23, wherein the outer member is made of a polymer that is reinforced with at least one of a fiber, braid, or webbing.

32. (canceled)

33. (canceled)

34. The medical device of claim 23 further comprising a conductor connected to the electrode, which conductor extends through the inner member to the proximal end portion, for enabling sensing of electrical activity of tissue in contact with the electrode.

35. The medical device of claim 23, wherein the inner member includes at least two lumens therein, a first lumen being capable of receiving a guide wire and a second lumen being capable of delivering fluids there through to the distal end portion of the inner member.

36. (canceled)

37. (canceled)

38. (canceled)

39. (canceled)

40. A medical device guide having an inner member having a proximal end, a distal end, and at least one lumen therein for the delivery of a medical device, and an outer member surrounding the proximal portion of the inner member such that the distal end of the inner member projects beyond the distal end of the outer member, at least one lumen having a section with one or more score lines along its length for the device being delivered, and the outer member having a slit that is aligned with the scoring in the inner member.

41. The medical device guide of claim 40, wherein the slit opening in the outer member is slightly larger than the inner member to permit easy removal of the medical device.

42. (canceled)

43. (canceled)

44. (canceled)

45. The medical device guide of claim 40, wherein the outer member is sufficiently stiff that the proximal end of the medical device guide can be engaged to be driven by a medical device advancer.

46. The medical device guide of claim 40, wherein the inner member includes a means of locking the medical device in place for coincident movement and of unlocking the medical device for independent movement.
